(19) United States
(12) Patent Application Publication
(10) Pub. No.: US 2023/0298751 A1
(43) Pub. Date: Sep. 21, 2023

(54) Prognosis Prediction Device and Program

(71) Applicant: The University of Tokyo, Tokyo (JP)

(72) Inventors: Kazuki TAOKA, Tokyo (JP); Ayumu TSUBOSAKA, Tokyo (JP)

(21) Appl. No.: 17/917,685

(22) PCT Filed: Mar. 16, 2021
(86) PCT No.: PCT/JP2021/010712

§ 371 Date: Apr. 7, 2023

(30) Foreign Application Priority Data
Apr. 10, 2020 (JP) 2020-070768

(51) Int. Cl. G16H 50/20; G16H 50/70

(52) U.S. Cl. CPC G16H 50/20; G16H 50/70

(57) ABSTRACT

A prognosis prediction device 1 includes a circuitry for receiving which receives a combination of information including known factor information including at least one type of clinical information and known prognosis information, and circuitry for machine learning which performs machine learning of at least one machine learning model by at least one machine learning algorithm such that the corresponding known prognosis information is outputted in response to an input of the received known factor information. A result of a machine learning is used in a process for predicting a prognosis of a prognosis prediction target patient.

### TECHNICAL FIELD

The present invention relates to a prognosis prediction device and a program for predicting a prognosis of a disease.

### RELATED ART

The number of elderly people is increasing in recent years. In order to secure beds and improve the efficiency of management thereof, a prediction of prognoses of elderly people's diseases has been required. For example, PTL 1 discloses an example of an apparatus for predicting the progression of a cancer.

### CITATION LIST

### Patent Literature

### [PTL 1]

JP-T-2009-537108

### SUMMARY

### Technical Problem

It has been known that a prognosis of a genetic disease such as a cancer can be predicted from genetic codes or the like, as described in PTL 1. However, a factor of, for example, pneumonia which is a major cause of deaths of elderly people has been not necessarily clear. Thus, it has been difficult to predict a prognosis of pneumonia.

The present invention has been made in view of the above circumstances, and one object thereof is to provide a prognosis prediction device and a program for predicting a prognosis of a disease such as pneumonia whose factor is unclear.

### Solution to Problem

One aspect of the present invention for solving the above problems in the conventional examples, is a prognosis prediction device including a circuitry for receiving which receives a combination of known information including factor information including at least one type of clinical information and prognosis information, and a circuitry for machine learning which performs machine learning of at least one machine learning model by at least one machine learning algorithm such that the corresponding known prognosis information is outputted in response to an input of the received known factor information, in which a result of a machine learning process performed by the circuitry for machine learning is used in a process for predicting a prognosis of a prognosis prediction target patient.

### Advantageous Effect of Invention

According to the present invention, a prognosis of a disease such as pneumonia whose factor is unclear can be predicted.

### BRIEF DESCRIPTION OF DRAWINGS

FIG. 1 is a block diagram illustrating a configuration example of a prognosis prediction device according to an embodiment of the present invention.

FIG. 2 is a functional block diagram illustrating an example of the prognosis prediction device according to the embodiment of the present invention.

FIG. 3 is a flowchart illustrating an example of a machine learning process in the prognosis prediction device according to the embodiment of the present invention.

FIG. 4 is a flowchart illustrating an example of an inference process in the prognosis prediction device according to the embodiment of the present invention.

FIG. 5 is an illustrative diagram of one example of an inference process in the prognosis prediction device according to the embodiment of the present invention.

### DESCRIPTION OF EMBODIMENT

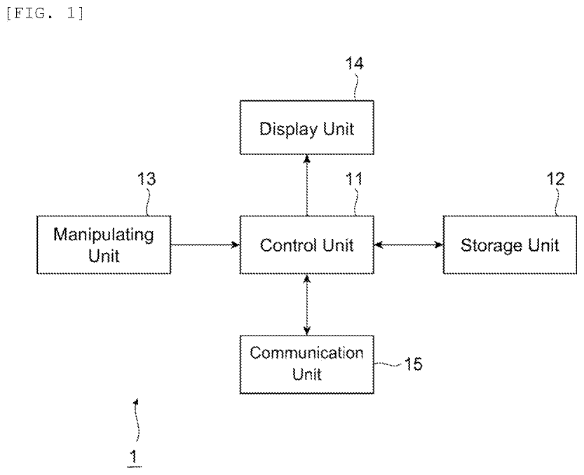

An embodiment of the present invention will be explained with reference to the drawings. One example of a prognosis prediction device 1 according to the embodiment of the present invention is a general computer apparatus including a control unit 11, a storage unit 12, a manipulating unit 13, a display unit 14, and a communication unit 15, as illustrated in FIG. 1.

The control unit 11 is a program control device such as a central processing unit (CPU). The control unit 11 operates in accordance with a program stored in the storage unit 12. In the present embodiment, the control unit 11 receives (a known information group including) known factor information and corresponding prognosis information, and selects, from among the factor information corresponding to the prognosis information, at least one type of factor information to become a major factor by using a model that outputs prognosis information in accordance with the received factor information. Here, the factor information includes at least one type of clinical information, for example. In one example, in addition to the clinical information, the factors include at least one of information regarding a medical test result, information regarding a medical history, information regarding a drug being currently taken, information for identifying a fungus or virus that may cause a disease (e.g. the presence/absence of a causative fungus or a resistant fungus), and information regarding an initial response.

The clinical information includes the age, the sex, the body height, the body weight, the body mass index (BMI), the number of times of hospitalizations, the place of residence (which may indicate whether or not the subject lives in a nursing home), or the race of a subject, and further, includes a performance status (PS), a body temperature, a blood pressure, an oxygenation index (e.g. an oxygen level (P/F)), a respiratory rate, and a heart rate which are vital information at the start of hospitalization (or at the start of treatment).

Further, the information regarding a medical test result includes white blood cell counts (WBC), hemoglobin (HB), and platelets (PLt), which are blood test information, an amount related to a nutritional status (e.g. an albumin (Alb) value), an amount related to a kidney function (e.g. a urea acid level (blood urea nitrogen (BUN)), a creatinine (Cre) level, an estimated glomerular filtration rate (eGFR)), an amount related to a liver function (glutamic-oxaloacetic transaminase (GOT), glutamic-pyruvic transaminase (GPT)), an amount related to the presence/absence of an inflammation or an infectious disease (e.g. a C-reactive protein (CRP) level), a total bilirubin level (T-bil), and a virus PCR test (of coronavirus or the like).

Moreover, the information regarding a medical history may indicate whether or not the subject has suffered from high blood pressure, diabetes, a circulatory disease, a chronic heart failure, cerebral infarction, a respiratory disease, sepsis, or a cancer (malignant tumor). The information regarding a drug being currently taken may identify the type or group of an antibiotic being currently taken, the dosage thereof, and the like.

In addition, the initial response information concerns a treatment effect to be exerted after the elapse of a prescribed time period from start of treatment. This information indicates a fever type (body temperature change) or a C-reactive protein (CRP) level during five to seven days from the start of treatment, for example.

By using, as training data, a combination of information including known factor information of the selected type and known prognosis information, the control unit 11 performs a machine learning process such that the prognosis information is outputted in response to an input of factor information of the selected type. Here, it is assumed that machine learning that is performed by the control unit 11 is decision tree analysis or random forest (L. Breiman: “Random Forests,” Machine Learning, 45, 1, pp. 5-32 (2001)) analysis based on the factor information, for example.

Specifically, in accordance with a prescribed model, the control unit 11 creates a decision tree (a regression tree or a classification tree) or a random forest using factor information of the type selected as a major factor. As a method of the machine learning process for creating a decision tree, a widely known method such as C4.5 may be used.

By using the decision tree or the random forest obtained as a result of the machine learning process, the control unit 11 performs a prognosis prediction process in which factor information regarding a prognosis prediction target patient is inputted. Factor information to be inputted here is factor information of the type selected in accordance with the above model. The details of operation of the control unit 11 will be explained later.

The storage unit 12 is a memory device or a disk device, for example. The storage unit 12 holds a program to be executed by the control unit 11. In addition, the storage unit 12 also serves as a work memory for the control unit 11.

The manipulating unit 13 is a mouse or a keyboard, for example. The manipulating unit 13 receives a user manipulation and outputs information indicated by the manipulation to the control unit 11. The display unit 14 is a display, for example. In accordance with a command inputted thereto from the control unit 11, the display unit 14 outputs and displays information.

The communication unit 15 is a network interface, for example. In accordance with a command inputted thereto from the control unit 11, the communication unit 15 exchanges various types of data with a personal computer, a tablet, or a smartphone which is an information terminal of an existing hospital or clinic, or with a cloud system, over a network.

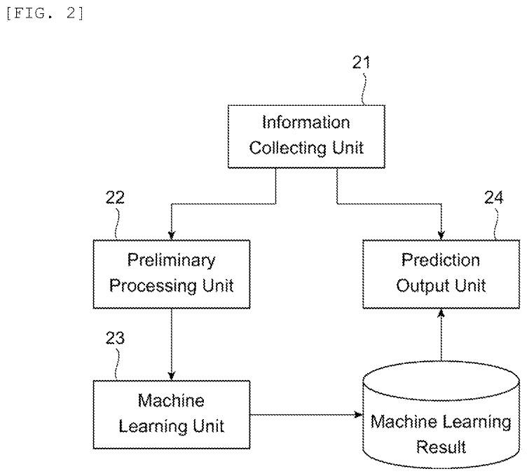

Next, exemplary operation of the control unit 11 of the present embodiment will be explained. In the present embodiment, the control unit 11 executes a machine learning process and a prediction process using a result of the machine learning process. In terms of the functionality, the control unit 11 includes an information collecting unit 21, a preliminary processing unit 22, a machine learning unit 23, and a prediction output unit 24, as illustrated in FIG. 2.

In the stage of the machine learning process, the information collecting unit 21 receives (a combination of known information including) known factor information and corresponding prognosis information. Specifically, this combination includes a plurality of information sets regarding past patients whose prognoses are known. The factor information includes information regarding a medical test result, information regarding a medical history, information regarding a drug being currently taken, information for identifying a fungus or virus that may cause a disease, and information regarding an initial response, as previously explained.

Further, prognosis information that is obtained by the information collecting unit 21 may include a plurality of types of prognosis information including prognosis information concerning the progress of a disease such as a hospitalization period (the number of days from admission to discharge) or the probability that the disease will become severe, a survival time (the number of days from admission to death), and prognosis information concerning the end of the disease indicating a possibility that the patient finally survives or dies.

Further, in the stage of the prediction process, the information collecting unit 21 receives factor information regarding a prognosis prediction target patient. The type of factor information that is required in the prediction process is selected in the course of the machine learning process, and the preliminary processing unit 22 outputs information indicating the selected factor information type, which will be explained later. Therefore, it is sufficient that the information collecting unit 21 collects, from among the factor information regarding the prognosis prediction target patient, factor information of the type selected in the machine learning process.

The preliminary processing unit 22 operates in the stage of the machine learning process. The preliminary processing unit 22 selects at least one type of information to become a major factor from among factor information corresponding to prognosis information by using a model that outputs prognosis information in accordance with factor information obtained by the information collecting unit 21.

Specifically, in one example of the present embodiment, the preliminary processing unit 22 uses, as the above model, a Cox proportional hazard model. That is, the preliminary processing unit 22 generates a relative risk function which is an exponential function of a linear combination of the factor information values, and obtains a p value (significance) and a hazard ratio β of each piece of the factor information by partial likelihood estimation or the like. A detailed explanation of this calculation will be omitted herein because this is widely known as an estimation method for a general Cox proportional hazard model.

The preliminary processing unit 22 identifies and selects factor information for which the obtained p value is less than a prescribed threshold (e.g. 0.05) (is significant). Then, the preliminary processing unit 22 outputs information (information for identifying the type of the factor information selected as a major factor such as information that identifies the type of the factor which indicates a PS as vital information) indicating the type of the identified factor information.

In addition, the preliminary processing unit 22 may output information indicating the type of factor information that is different from the identified factor information but is designated by a user which is considered to be clinically important for the user as well as the information indicating the identified factor information type.

The machine learning unit 23 operates in the stage of the machine learning process. The machine learning unit 23 performs machine learning to obtain a predetermined decision tree or a predetermined random forest such that corresponding prognosis information is outputted as an objective variable in response to an input of factor information, from among the factor information obtained by the information collecting unit 21, identified by the information outputted from the preliminary processing unit 22.

Here, as a method for the machine learning process which is used to create a decision tree or a random forest, a widely known method such as C4.5 may be used, as previously explained. In this case, a hyperparameter of the decision tree or random forest which is a result of the machine learning may be empirically set. Otherwise, in order to set a hyperparameter, a method for optimizing hyperparameters by trial and error without any manual operation (e.g. optuna (https://optuna.org)), by executing multiple machine learning processes in parallel with each other with use of multiple sets of hyperparameters, and selecting a set of hyperparameters whose learning curve (variation of the performance for generalizing machine learning results with respect to an increase in the number of training data items) is the most preferable, for example.

For example, to obtain a decision tree as a machine learning result by means of the machine learning unit 23, a maximum depth value (max depth), a maximum leaf node value (max leaf nodes), and a determination criteria (Gini or entropy) which are hyperparameters of the decision tree are preliminarily decided in an empirical manner or by trial and error.

As a result of the process performed by the machine learning unit 23, machine learning of the relation between factor information of the type decided as a major factor by the preliminary processing unit 22 and a prognosis is conducted, and prognosis information can be inferred on the basis of the factor information.

The prediction output unit 24 operates in the stage of the prediction process. The prediction output unit 24 receives information (which may be an identifier or a name, for example) for identifying a prognosis prediction target patient and factor information regarding the patient obtained by the information collecting unit 21. The prediction output unit 24 predicts a prognosis by using a machine learning result (a decision tree or a random forest) obtained by the machine learning unit 23 and factor information, among the received factor information, used by the machine learning unit 23, and outputs the predicted prognosis information. For example, when prognosis information is information regarding a hospitalization period (the number of days from admission to discharge) and a survival time (the number of days from admission to death), the prediction output unit 24 estimates the information (in a case where a hospitalization period is estimated, there is no information regarding a survival time, and, if a survival time is estimated, a hospitalization period is identical to the survival time) on the basis of the factor information regarding the prognosis prediction target patient by using the decision tree or the like created by the machine learning unit 23, and outputs a prediction result of the prognosis as well as the information for identifying the patient.

Here, an output from the prediction output unit 24 may be given to the abovementioned display unit 14, or may be sent to separate systems via the communication unit 15 to be displayed at the separate systems. The separate systems include any other computer devices such as a personal computer, a tablet computer, and a smartphone.

Alternatively, the separate systems may include an electronic medical record system, a nurse call system, and any types of terminal devices for medical workers.

After receiving information regarding a predicted prognosis from the prediction output unit 24, the separate systems display the information by means of respective display means.

### [Checking Progress Information]

In the abovementioned examples of the present embodiment, factor information which may be considered to be clinically important for a user and are used in machine learning and an inference process using a machine learning result may include factor information (information regarding an initial response) concerning a treatment effect to be exerted after the elapse of a prescribed time period from the start of treatment.

In this example, the machine learning unit 23 receives factor information concerning a treatment effect to be exerted after the elapse of a predetermined time period from the start of treatment, and performs a machine learning process to output prognosis information in response to an input of the factor information together with other factor information (factor information of the type selected by the preliminary processing unit 22).

Thereafter, the prediction output unit 24 provides a result of the machine learning process for a process of predicting a prognosis of the prognosis prediction target patient.

Specifically, factor information concerning a treatment effect to be exerted after the elapse of a predetermined time period from the start of treatment includes a fever type (body temperature change) or a C-reactive protein (CRP) level during five to seven days from the start of treatment, as previously explained. In the prognosis prediction device 1 of the present embodiment, each time such information regarding a response of the prognosis prediction target patient to treatment is obtained, the prediction output unit 24 updates a predicted prognosis of the prognosis prediction target patient by using inputted information including the obtained factor information and the decision tree or the like created as a result of the machine learning process by the machine learning unit 23, and then, outputs information indicating the updated result of the predicted prognosis. This output also may be given to the display unit 14, or may be sent to separate systems via the communication unit 15 to be displayed at the separate systems in the same manner as that explained above.

### [Regional Difference]

It has been known that there is a regional difference in infectious diseases. For example, regarding germs, pneumonia is caused mainly by Pseudomonas aeruginosa in some areas while pneumonia is caused mainly by Streptococcus pneumoniae in other areas. Also, the sensitivity to causative germs varies from area to area due to the difference in the use frequency of antibiotics. Furthermore, regarding what is called the new coronavirus (SARS-CoV-2) which is being spread in the year 2020, it is pointed out that viruses created by different mutations are spread in different areas.

Therefore, to predict a prognosis of a regional disease such as an infectious disease, the information collecting unit 21 of the prognosis prediction device 1 of the present embodiment obtains a combination (a combination of known information) of factor information which is training data and prognosis information for each of the areas in which patients live while taking the regional difference into consideration. Then, on the basis of the combination of known information obtained for the respective areas, the prognosis prediction device 1 selects respective major factor information, executes machine learning processes, and thereby creates decision trees or the like which are machine learning results for the respective areas.

In the present example, the prognosis prediction device 1 predicts a prognosis by using a decision tree or a random forest obtained for the residence area of a prognosis prediction target patient and factor information regarding the prognosis prediction target patient, and outputs the predicted prognosis information. It is to be noted that an administrative district such as a prefecture may be set as the area. The range of such an area may be empirically defined.

### [Operation]

The operation of the present embodiment which has the above configuration is as follows. In order to use the prognosis prediction device 1 of the present embodiment, a combination of known information including factor information regarding past patients who have been hospitalized in hospitals located in one or more areas including the area (e.g. prefecture) in which a prognosis target patient lives, and the corresponding prognosis information is preliminarily prepared.

It is to be noted that, in the following example, the prognosis prediction device 1 predicts a prognosis of pneumonia of an elderly person. In this example, the factor information includes clinical information, information regarding a medical test result, information regarding a medical history, information regarding a drug being currently taken, information regarding a fungus or virus that may cause the disease, the presence/absence of a disease resistant fungus, and information regarding an initial response. It is assumed that a hospitalization period (the number of days from admission to discharge) or a survival time (the number of days from admission to death) is used as prognosis information.

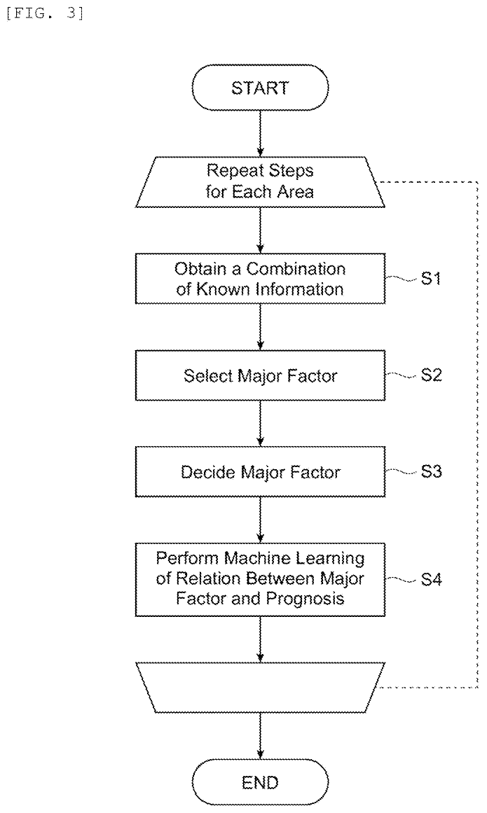

First, the prognosis prediction device 1 executes the machine learning process. In this stage, the prognosis prediction device 1 obtains a combination of prepared and known information for each corresponding area (hereinafter, referred to as “process target area”) (S1), as illustrated in FIG. 3. Then, the prognosis prediction device 1 obtains a p value (significance) and a hazard ratio β of each type of factor information on the basis of factor information according to the combination of obtained known information, by partial likelihood estimation or the like using a Cox proportional hazard model that outputs corresponding prognosis information. The prognosis prediction device 1 identifies and selects, as a major factor, factor information whose p value obtained here is less than a predetermined threshold (e.g. 0.05) (is significant) (SELECT MAJOR FACTOR: S2).

In addition, the prognosis prediction device 1 obtains (information regarding the type of factor information included in either one of) information indicating a factor information type (for example, information regarding an initial response) preliminarily designated as clinically important information by a user, and information indicating the factor information type selected at step S2 (DECIDE MAJOR FACTOR: S3). The information indicating the factor information types is stored in association with information for identifying a process target area.

The prognosis prediction device 1 uses, as training data, the combination of known information obtained at step S1, and performs machine learning to obtain a random forest such that the corresponding prognosis information is outputted as an objective variable in response to an input of factor information, among the factor information, of the type identified by the information obtained at step S3 (S4).

The prognosis prediction device 1 repeats the process of steps S1 to S4 for each of the areas according to the prepared combination of known information, obtains machine learning results which are random forests for the respective areas, and stores the machine learning results in association with information for identifying the process target area.

Accordingly, the prognosis prediction device 1 holds information for identifying an area, information indicating the type of factor information regarded as a major factor, and information indicating a corresponding machine learning result (information for identifying a corresponding random forest) that are associated with one another.

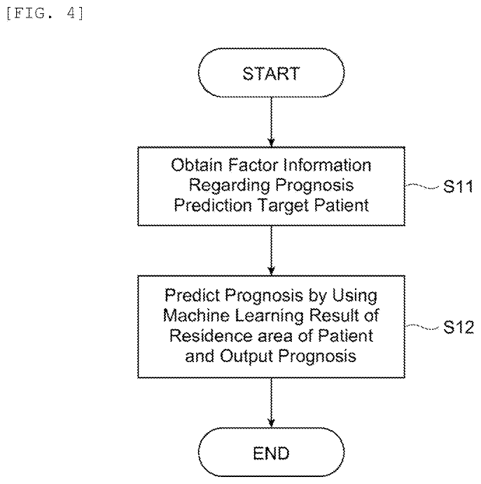

Next, a prediction process using the prognosis prediction device 1 will be explained. In the stage of executing the prediction process, the prognosis prediction device 1 receives factor information regarding a patient designated as a prognosis prediction target by a user (S11), as illustrated in FIG. 4. Only factor information of a type that is stored in association with the residence area of the prognosis prediction target patient and that is regarded as a major factor may be received. In addition, it is not necessary that information regarding an initial response is present at first. In a case where there is no factor information of a certain type, the prognosis prediction device 1 considers that such factor information is a missing value, and executes the following steps.

The prognosis prediction device 1 obtains a prediction result of prognosis information (S12) by using the factor information received at step S11 and a random forest that has been subjected to machine learning and stored in association with information for identifying the residence area of the prognosis prediction target patient.

Since it is assumed that a hospitalization period (the number of days from admission to discharge) and a survival time (the number of days from admission to death) are prognosis information, the prognosis prediction device 1 estimates and outputs a hospitalization period or a survival time (in a case where a hospitalization period is estimated, there is no information regarding a survival time, and, if a survival time is estimated, a hospitalization period is equal to the survival time).

It is to be noted that, in order to estimate an objective variable from information including a missing value by using a random forest or the like, a variety of widely known methods including a method in which the missing value is substituted by a representative value, and a method in which the missing value is estimated and used can be used. An explanation thereof will be omitted herein.

The prognosis prediction device 1 repeatedly executes the abovementioned process every prescribed number of days (e.g. five days or seven days) as long as the prognosis prediction target patient is alive, and updates a predicted prognosis of the prognosis prediction target patient.

[Example in which Preliminary Process is not Executed]

In another example of the prognosis prediction device 1 of the present embodiment, the prognosis prediction device 1 may skip the preliminary process, and perform the machine learning process based on a preliminarily selected factor information. In this example, the preliminary processing unit 22 implemented with the control unit 11 outputs, as information for identifying the type of major factor information, information for identifying at least one preliminarily selected factor information, and the machine learning unit 23 performs machine learning to obtain a decision tree or a random forest to output, as an objective variable, the corresponding prognosis information in response to an input of factor information, among the factor information obtained by the information collecting unit 21, of the type identified by the information outputted from the preliminary processing unit 22.

In some cases, the machine learning unit 23 performs a machine learning process of sub-sampling factor information, or performs a machine learning process of determining the importance of factor information. In these cases, it is not necessary to select factor information that is regarded as a major factor in the stage of the preliminary process.

In these examples, operation of the prediction output unit 24, which operates in the stage of the prediction process, is as follows. The prediction output unit 24 receives information (which may be an identifier or a name, for example) for identifying a prognosis prediction target patient and factor information regarding the target patient obtained by the information collecting unit 21. Then, the prediction output unit 24 predicts a prognosis by using a machine learning result which is a decision tree or a random forest obtained by the machine learning unit 23 and factor information, among the received factor information, used in the machine learning (used in a prediction process as a result of machine learning if sub-sampling is conducted) by the machine learning unit 23, and outputs the predicted prognosis information.

### [Another Example of Machine Learning]

In the explanation given so far, a machine learning result created by the control unit 11 operating as the machine learning unit 23 is a common decision tree or a common random forest. However, the present embodiment is not limited to this case. XGBoost or Light GBM (Gradient Boosting) may be used, or any other deep learning model may be used. Also in this case, it is sufficient that a hyperparameter of each model is decided in an empirical manner or by try and error using optuna or the like.

### [Example of Selecting Machine Learning Model and Algorithm]

In the explanation given so far, the machine learning unit 23 is configured to perform machine learning of a predetermined decision tree or random forest. In another example of the present embodiment, an effective model or algorithm may be selected and used from among a plurality of machine learning models or machine learning processes.

In one example, the machine learning unit 23 operates in the stage of the machine learning process, and performs machine learning of a plurality of preliminarily selected machine learning models through the corresponding machine learning process such that the corresponding prognosis information is outputted as an objective variable in response to an input of, among factor information obtained by the information collecting unit 21, factor information identified by information outputted from the preliminary processing unit 22 or factor information of a predetermined type.

The preliminarily selected machine learning processes may include a variety of decision trees or classifiers such as cat boost (Liudmila Prokhorenkova, et al., CatBoost: unbiased boosting with categorical features, arXiv:1706.09516v5), Light GBM (Gradient Boosting Machine:Guolin Ke, et al., Light GBM: A Highly Efficient Gradient Boosting Decision Tree), GBM, Extreme Gradient Boosting (XGBoost), ExtraTrees (Pierre Geurts, et al., Extremely randomized trees, Mach. Learn 63, 3-42(2006)), a random forest, Ada Boost Classifier, logistic regression, linear discriminant analysis (LDA), Naive Bayes, the K-nearest neighbor, the ridge classifier, and the support-vector machine, for example. It is to be noted that a hyperparameter of the model may be defined empirically, or may be defined by optuna or the like, which has been previously explained.

The machine learning unit 23 performs machine learning of the selected machine learning models through the corresponding machine learning processes as described above, and evaluates the machine learning results by using a combination of information including known factor information and known prognosis information. A detailed explanation of this evaluation is omitted because a widely-known method can be used therefor. For example, this evaluation may be made on the basis of an area under curve (AUC) value or an accuracy which are related to prognosis information.

The machine learning unit 23 arranges the selected machine learning models in the decreasing order of the AUC values or accuracies, and selects, as a learned model, a machine learning model arranged at the top (having the highest AUC value or accuracy).

For example, in a case where the order in which a plurality of machine learning processes are arranged is obtained as a result of the arrangement in the descending order of AUC values or accuracies, the order of CatBoost, Light GBM, GBM, Extreme Gradient Boosting (XGBoost) . . . , the machine learning unit 23 selects CatBoost whose machine learning result having the highest AUC value or accuracy as a learned model to use.

In this example, in the stage of the prediction process, the prediction output unit 24 performs a process using the machine learning result selected as a learned model by the machine learning unit 23 as follows. That is, the prediction output unit 24 of this example receives information for identifying a prognosis prediction target patient and factor information regarding the target patient obtained by the information collecting unit 21, and inputs the received factor information to a machine learning result, which is a machine learning result obtained by CatBoost in the abovementioned example, for example, selected as a learned model by the machine learning unit 23. Accordingly, predicted prognosis information is obtained. Then, the prediction output unit 24 outputs the predicted prognosis and the information for identifying the patient received simultaneously with the inputted factor information.

In the present example of the present embodiment, a prediction based on factor information can be made using a machine learning result having relatively high AUC and accuracy.

### [Plurality of Predicted Prognoses]

In a certain example of the present embodiment, prognosis information to be predicted may include a plurality of types of prognosis information including prognosis information concerning the progress of a disease and prognosis information concerning the end of a disease, as previously explained. Prognosis information regarding the progress of a disease indicates the probability of developing a severe symptom, or indicates whether an artificial respirator is required or not, or the presence/absence of the probability of entering an intensive care unit, for example. In addition, prognosis information concerning the end of a disease indicates whether or not the patient will highly probably die, for example.

In this example, the machine learning unit 23 may obtain machine learning results for respective types of prognosis information to be predicted. That is, the machine learning unit 23 may input a plurality of types of factor information, and perform machine learning of a first decision tree by CatBoost using prognosis information (for example, information represents a symptom from among a mild symptom, a moderate symptom, or a severe symptom, after the elapse of a prescribed number of days) concerning the progress of a disease as teacher information, and further, may input a plurality of types of factor information, and perform machine learning of a second decision tree by CatBoost using prognosis information (for example, whether a patient will survive or die after the elapse of a prescribed number of days) concerning the end of the disease.

It is to be noted that a combination of factor information types for use in machine learning of the second decision tree may be different from that used in machine learning of the first decision tree. That is, the preliminary processing unit 22 selects a (combination of) major factor information for each type of prognosis information to be predicted, and outputs information for identifying the type of the selected factor information.

When factor information of the type same as that used in machine learning is inputted to the first decision tree which is a result of the machine learning, the first decision tree may output the corresponding probability (score) of developing severe symptoms. In addition, factor information of the type same as that used in the machine learning is inputted to the second decision tree, the second decision tree outputs the corresponding case fatality rate (score).

That is, in this example, the prediction output unit 24 receives information for identifying the prognosis prediction target patient and factor information regarding the target patient obtained by the information collecting unit 21, and then, inputs, to the first decision tree which is a machine learning result obtained by machine learning performed by the machine learning unit 23, factor information, among the received factor information, used for machine learning of the first decision tree by the machine learning unit 23, and the probability of developing a severe symptom of the prognosis prediction target patient is predicted and outputted.

Further, the prediction output unit 24 inputs factor information, among the received factor information, used in machine learning of the second decision tree by the machine learning unit 23, to the second decision tree which is a machine learning result obtained by machine learning performed by the machine learning unit 23, and the probability that the prognosis prediction target patient will die is predicted and outputted.

The prediction output unit 24 may further plot a point group of outputs (the severe-symptom developing rate and the case fatality rate) from the prediction output unit 24 based on the combination of known factor information and prognosis information such that the severe-symptom developing rate and the case fatality rate are respectively indicated by intersecting axes, whereby a closed curve surrounding a point group concerning patients who have actually developed serious symptoms and a closed curve surrounding a point group concerning patients who have not developed any serious symptom can be obtained. In addition, a closed curve surrounding a point group concerning deceased patients may be generated. These closed curves may be generated by user operation or may be obtained by generating protruding hulls surrounding the corresponding point groups.

The prediction output unit 24 plots a point corresponding to an inference result for the prognosis prediction target patient on the same coordinate axes, and, if the inferred result belongs to either one of the closed curves, outputs information concerning the closed curve.

For example, in a case where a point corresponding to an inference result for the prognosis prediction target patient is plotted on a coordinate belonging inside the closed curve surrounding the point group concerning patients who have not developed severe symptoms, the prediction output unit 24 outputs a prediction indicating that the prognosis prediction target patient “will not develop a severe symptom.”

According to this example of the present embodiment, a group of patients who will not develop severe symptoms can be determined so that whether hospitalization is required or not can be determined. Also, a group of patients who will highly probably develop severe symptoms or who will highly probably die can be determined so that whether the prognosis prediction target patient requires hospitalization or not can be easily determined.

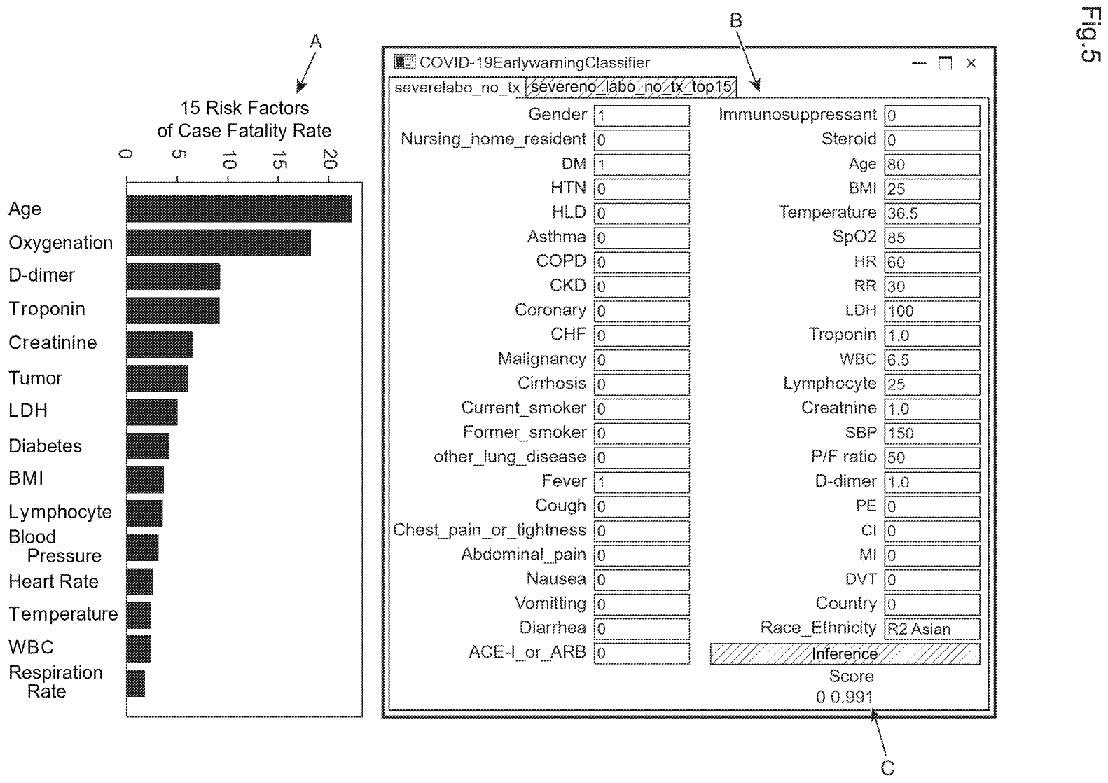

Further, in a certain example of the present embodiment, when a type of prognosis information (for example, either the probability of developing a severe symptom or the case fatality rate) to be predicted is selected, as illustrated in FIG. 5, the prediction output unit 25 displays the information (A) indicating factor information types that are identified as major factors in order to predict prognosis information of the selected type as a result of a process performed by the preliminary processing unit 22 or the machine learning unit 23, and also displays a field (B) in which factor information of at least the identified type (factor information used in machine learning) is inputted.

An input field for factor information of the type identified as a major factor may be displayed alone, or an input field for any other factor information (for example, an input field for factor information of a type included in a logical sum of a combination of major factor information identified for respective types of prognosis information which can be predicted) may also be displayed in addition to the factor information of the type identified as a major factor such that the input field for the factor information of the type identified as major factor information corresponding to prognosis information to be predicted and the other input field are distinguishable.

It is to be noted that major factor information may be factor information determined as major factor information as a result of a preliminary process or a machine learning process, or may be factor information decided to be used in a prediction process as a result of machine learning if sub-sampling is performed during the machine learning.

Furthermore, in a case where, in the field (B), there is no input of information in the input field corresponding to any of the factor information of the types identified as major factors corresponding to prognosis information to be predicted, the prognosis prediction device 1 may display a description thereof so as not to perform a prognosis prediction process.

When factor information of the type identified as a major factor is inputted in the abovementioned displayed field (B) so as to correspond to prognosis information to be predicted, the prognosis prediction device 1 obtains a prediction result of the prediction target prognosis information by executing a process of the prediction output unit 24, and outputs the prediction result (C).

### [Analysis of Effects of Medicine]

In addition, the prognosis prediction device 1 of the present embodiment can determine the respective probabilities belonging to a mild symptom, a moderate symptom, and a severe symptom, as previously explained. Accordingly, patients are classified into a mild symptom group, a moderate symptom group, and a severe symptom group, some groups of patients belonging to each of the symptom groups are treated with different medicines, and the progress thereof is observed. Accordingly, the effects of the medicines can be analyzed.

For example, patients who are predicted to develop severe symptoms are divided into two groups, medicine A is administered to one of the groups while the medicine A is not administered to the other group. In this case, if the actual rate of developing severe symptoms of the one group is determined to be significantly lower than that of the other group, it can be confirmed that the medicine A is effective for a disease from which the patients of the one group suffer.

[Extraction of Information from Electronic Medical Record]

The prognosis prediction device 1 of the present embodiment may collaborate with what is called an electronic medical record system, or may be implemented as a partial function of the electronic medical record system. In the present example, the prognosis prediction device 1 extracts, from the electronic medical record system, training data for a machine learning process or factor information regarding a prognosis prediction target patient for an inference process, and uses the extracted information for these processes.

Further, in this example, a prediction result of a prognosis outputted from the prognosis prediction device 1 may be outputted and displayed in the electronic medical record system in a manner such as that previously explained.

[Example of being Implemented as Server]

Alternatively, the prognosis prediction device 1 of the present embodiment may be implemented as a server. In this case, when receiving an access made by an external computer system such as an electronic medical record system, the prognosis prediction device 1 receives training data for machine learning, information regarding a prognosis prediction target patient (information for identifying the patient, information for identifying the place of residence, and factor information regarding the patient, etc.) from the external computer system, and executes a machine learning process and an inference process.

After performing the inference process, the prognosis prediction device 1 of this example outputs a prediction result of the prognosis to an output destination designated by the external computer system. The output destination may be an electronic medical record system, a nurse call system, or a terminal device for medical workers, for example.

### Advantageous Effects of Embodiment

According to the present embodiment, even in a case where a factor that has an influence on a prognosis of a disease such as pneumonia of an elderly person who has not taken a clinical test is unknown, a treatment guide using what is called real world data can be determined, and further, a prognosis of the disease can be predicted.

### REFERENCE SIGNS LIST

1: Prognosis prediction device
11: Control unit
12: Storage unit
13: Manipulating unit
14: Display unit
15: Communication unit
21: Information collecting unit
22: Preliminary processing unit
23: Machine learning unit
24: Prediction output unit

1. A prognosis prediction device comprising:
a circuitry for receiving which receives a combination of known information including factor information including at least one type of clinical information and prognosis information; and
a circuitry for machine learning which performs machine learning of at least one machine learning model by at least one machine learning algorithm such that corresponding known prognosis information is outputted in response to an input of the received known factor information, wherein
a result of a machine learning process performed by the machine learning means is used in a process for predicting a prognosis of a prognosis prediction target patient.

2. The prognosis prediction device according to claim 1, further comprising:
circuitry for preliminary processing which processes selecting, from among factor information corresponding to prognosis information, at least one type of factor information to become a major factor, in accordance with a model that outputs prognosis information, by using factor information including clinical information, information regarding a test result, information regarding a medical history, information regarding a drug being currently taken, and information for identifying a fungus or virus that may cause a disease, wherein,
by using a combination of information including known factor information of the type selected by the circuitry for preliminary processing and known prognosis information, the circuitry for machine learning performs a machine learning process of performing machine learning of at least one machine learning model by at least one machine learning algorithm such that the corresponding known prognosis information is outputted in response to an input of the known factor information of the type selected by the circuitry for preliminary processing.

3. The prognosis prediction device according to claim 2, wherein
the circuitry for preliminary processing uses, as the model, a Cox proportional hazard model.

4. The prognosis prediction device according to claim 1, wherein
the circuitry for machine learning further receives, as factor information, initial response information which relates to a treatment effect to be exerted after elapse of a prescribed time period from start of treatment, and performs the machine learning process such that prognosis information is outputted in response to an input of the initial response information, and
a result of the machine learning process is used in a process for predicting a prognosis of a prognosis prediction target patient.

5. The prognosis prediction device according to claim 4, further comprising:
a circuitry for obtaining which obtains, for the prognosis prediction target patient, initial response information which concerns a treatment effect to be exerted after elapse of a prescribed time period from the start of treatment, wherein,
each time the initial response information is obtained, a prediction of a prognosis of the prognosis prediction target patient is updated with the use of inputted information including the obtained initial response information and a result of the machine learning process performed by the circuitry for machine learning.

6. The prognosis prediction device according to claim 1, wherein
the prognosis information includes prognosis information concerning the progress of a disease and prognosis information concerning an end of a disease,
the circuitry for machine learning performs machine learning of at least one machine learning model by at least one machine learning algorithm such that the corresponding known prognosis information concerning the progress of a disease and the corresponding known prognosis information concerning the end of a disease are outputted in response to an input of the received known factor information, and
a result of the machine learning process performed by the circuitry for machine learning is used in a process for predicting a prognosis concerning the progress of the disease of the prognosis prediction target patient and a prognosis concerning the end of the disease of the prognosis prediction target patient.

7. A non-transitory computer readable medium storing a program for causing a computer to:
preliminary process for selecting, from among factor information corresponding to prognosis information, at least one type of factor information to become a major factor, in accordance with a model that outputs prognosis information, by using factor information including clinical information, information regarding a test result, information regarding a medical history, information regarding a drug being currently taken, and information for identifying a fungus or virus that may cause a disease;
perform a machine learning process of performing machine learning of at least one machine learning model by at least one machine learning algorithm by using a combination of information including known factor information of the type selected by the preliminary process means and known prognosis information, such that corresponding known prognosis information is outputted in response to an input of known factor information of the type selected by the preliminary processing means; and
perform a prognosis prediction process in which, for a prognosis prediction target patient, known factor information of the type selected by the preliminary processing means is inputted information based on a result of the machine learning process.

8. A non-transitory computer readable medium storing a program for causing a computer to:
receive a combination of information including known factor information including at least one type of clinical information, known prognosis information concerning progress of a disease, and known prognosis information concerning an end of the disease;
perform machine learning of at least one machine learning model by at least one machine learning algorithm such that corresponding known prognosis information concerning progress of the disease and corresponding known prognosis information concerning the end of the disease are outputted in response to an input of the received known factor information; and
execute a prediction process of a prognosis concerning progress of a disease of a prognosis prediction target patient and a prognosis concerning an end of the disease of the prognosis prediction target patient on a basis of a result of the machine learning process.
